(19) United States
(12) Patent Application Publication
(10) Pub. No.: US 2026/0272707 A1
(43) Pub. Date: Sep. 17, 2026

(54) HEAT TRANSFER UNIT, APPARATUS AND ASSOCIATED METHOD

(71) Applicant: HILOTHERM HOLDING AG, Oberwil b. Zug (CH)

(72) Inventor: Christian STEGMANN, Burgberg im Allgäu (DE)

(21) Appl. No.: 19/162,841

(22) PCT Filed: Mar. 8, 2024
(86) PCT No.: PCT/EP2024/056123

§ 371 Date: Sep. 5, 2025

(30) Foreign Application Priority Data
Mar. 9, 2023 (LU) LU503597

(51) Int. Cl. A61F 7/02; A61F 7/00

(52) U.S. Cl. CPC A61F 7/02; A61F 2007/0003; A61F 2007/0056; A61F 2007/0231; A61F 2007/0257; A61F 2007/0274

(57) ABSTRACT

A heat transfer unit adapted to be applied to a human face in order to transfer heat to/from the human face comprises a first flexible material layer and a second flexible material layer defining a fluid chamber therebetween, allowing a heat transfer fluid to flow through the fluid chamber. The first and second flexible material layers are connected to one another at a plurality of connection locations. The heat transfer unit is dimensioned such that, when applied to a human face, it can cover at least 70% of the surface of the human face. The heat transfer unit can be used to heat or cool a human face, in particular in a thermal therapy treatment of a human face, and in particular in the treatment of tissue damage after surgical interventions, injuries or inflammations.

This application claims priority to Luxemburg Patent Application No. LU 503597 filed Mar. 9, 2023, the entire contents of which are hereby incorporated by reference.

The present invention relates to a heat transfer unit, a face cuff assembly and a heat transfer apparatus for use in the thermal treatment of a human face, in particular for use in the thermal therapy treatment of a human face, and in particular for use in the treatment of tissue damage after surgical interventions, of injuries, of inflammations, as well as a method of heating or cooling a human face.

In the context of the present invention, the terms “thermal treatment” and “thermal therapy treatment” are intended to be understood to mean a treatment in which heat is supplied to the human face, or removed therefrom.

Preferably, this is not a matter of supplying heat to, or removing heat from, the entire body, as is done, for example, in a sauna and the subsequent immersion in a plunge pool filled with cold water, but it is preferably a matter of supplying heat specifically to, or removing heat specifically from, the face, in order to achieve an effect, in particular a therapeutic effect, preferably without significantly supplying heat to, or removing heat from, the remainder of the human body.

WO2022/101320 discloses a heat transfer unit suitable for being applied to various regions of the human body, as well as a heat transfer apparatus.

It is an object of the present invention to provide an alternative heat transfer unit and heat transfer apparatus, in particular a further improved heat transfer unit and heat transfer apparatus, as well as an associated face cuff assembly and associated method.

Aspects of the present invention are set out in the claims. Each feature disclosed and/or illustrated in the present specification may be incorporated in the invention, whether alone or in combination with any other feature disclosed or illustrated herein, unless such a combination is explicitly excluded or technically impossible. This relates to apparatus aspects and method aspects alike, as well as their preferred embodiments. Accordingly, for example, any of the devices disclosed herein can be used in the context of any of the methods disclosed herein, and vice versa.

According to a first aspect, the present invention provides a heat transfer unit adapted to be applied to a human face in order to transfer heat to/from the human face,
wherein the heat transfer unit comprises a first flexible material layer and a second flexible material layer defining a fluid chamber therebetween, allowing a heat transfer fluid to flow through the fluid chamber,
wherein the first and second flexible material layers are connected to one another at a plurality of connection locations,
wherein the heat transfer unit is dimensioned such that, when applied to a human face, it can cover at least 70% of the surface of the human face.

The present inventor has appreciated that existing heat transfer units tend to cover only a relatively small portion of the human face, such as the eye region, e.g. a region between a first approximately horizontal line located about 2 or 3 cm above the eyes and a second approximately horizontal line located about 2 or 3 cm below the eyes. He has further appreciated that it would be useful to provide a heat transfer unit that is able to cover a larger proportion of the human face.

In the sense of the invention, the term “human face” is preferably intended to be understood to mean a human face of average size and shape, in particular of an adult. Further, for the purpose of the present application, a “human face” is preferably intended to be understood to relate to the front part of a human head reaching to the hairline and further including the ears, cheeks and chin, either reaching the front of the chin, or including the portion of the human head extending in a rearward direction from the front of the chin up to, but excluding, the neck.

Preferably, when applied to a human face, the heat transfer unit is in sufficiently close contact to the human face (or a large proportion thereof) to ensure efficient heat transfer.

The first and second flexible material layers may be substantially fluid tight so as to form a substantially fluid tight fluid chamber (apart from any external connections described below). The first and second flexible material layers may be made from the same, or different materials, for example plastics material such as low-density polyethylene (LDPE). Other examples include polyurethane. The first and second flexible material layers may, for example, have a thickness of at least 0.1, 0.15, 0.2 or 0.25 mm and/or at most 0.5, 0.45, 0.4, 0.35 or 0.3 mm.

Since the first and second materials are flexible, the heat transfer unit can be at least partially wrapped around the face and adapt to its individual shape to result in at least partial contact of the heat transfer unit, in particular the first flexible material layer, with the face. At least some embodiments can enable better, or more complete, contact between the heat transfer unit and the face than has been possible with prior art heat transfer units.

When a heat transfer fluid that is warmer than the face is caused to flow through the fluid chamber, heat is transferred to the face, thereby heating the face. When a heat transfer fluid that is colder than the face is caused to flow through the fluid chamber, heat is transferred from the face, thereby cooling the face. The latter may be employed in particular after surgical interventions affecting the face, in particular facial feminization surgery or facial masculinization surgery.

The connection locations at which the first and second material layers are connected to one another may be individual, relatively small, regions and may, for example, be substantially round or of any other suitable shape. The connection locations are sometimes also referred to as “dots”.

In order to connect the first and second material layers at the connection locations, the first and second material layers may be fused or welded together at selected locations. Alternatively, adhesive material may be used to connect the first and second material layers at the connection locations. Suitable methods of connecting the first and second material layers are, in principle, known to those skilled in the art and will not be described in detail.

In addition to being connected at the (individual) connection locations, the first and second material layers are connected at their periphery, in particular all along their periphery, with the possible exception of any external fluid connections—although any external fluid connections may alternatively be provided only in the first and/or second material layer, in which case the first and second material layers may be connected all along their periphery without interruption. The first and second material layers may be connected at their periphery using the same or a similar technique as the technique used for connecting the first and second material layers at the connection locations. In particular, the joining of the first and second material layers at the connection locations and along their periphery may be performed simultaneously, in particular using the same processing apparatus.

The connection locations serve to locate the first and second material layers with respect to one another. Individual spaces or passageways for the heat transfer fluid are formed between the connection locations, as well as between the connection locations and the periphery of the heat transfer unit. Accordingly, the connection locations should be kept relatively small, so as not to obstruct unduly the flow of the heat transfer fluid. On the other hand, the connection locations may need to be of sufficient size to ensure a firm connection between the first and second material layers.

The average spacing between the connection locations in the first and second regions can be determined in a variety of ways. For example, a square or round area of the heat transfer unit (or of the first and second flexible material layers) can be considered, whereby this area should have at least 4, 5, 6 or more connection locations. For each connection location, the nearest neighbor or neighbors (nearest neighboring connection location or locations) should be determined, as well as the distance between the respective connection location and its nearest neighbor(s). The average of these distances then indicates the average spacing between the connection locations in that region. Alternatively, the average density of connection locations within a particular area could be determined, for example by determining the number of connection locations in the respective area of the heat transfer unit, or the total surface area occupied by the connection locations in the respective area of the heat transfer unit. The number of connection locations in the respective area of the heat transfer unit or the total surface area occupied by the connection locations in the respective area of the heat transfer unit can then be put in relation to the surface area under consideration so as to provide a measure of the average spacing between the connection locations in the respective area of the heat transfer unit. Other ways of determining the average spacing between the connection locations may be conceivable.

The connection locations may, for example, have a diameter of at least 2, 3 or 4 mm and at most 10, 9, 8, 7 or 6 mm. A preferred diameter may be about 5 mm, e.g. 5 mm+/−0.5 mm. The distance between nearest neighbors of connection locations (center to center) may, for example, be at least 10, 12 or 14 mm and at most 25, 20, 18 or 16 mm. A preferred distance (center to center) may be about 15 mm, e.g. 15 mm+/−0.5 mm. The connection locations may, for example, form a regular or irregular pattern, or a combination of regular and irregular patterns. In a regular pattern, the connection locations may, for example, be arranged such that three nearest neighbors of connection locations form an equilateral triangle, or such that four nearest neighbors of connection locations form a square.

In one embodiment, the heat transfer unit is dimensioned such that, when applied to a human face, it can cover at least 75%, 80%, 85% or 90% of the surface of the human face. A greater percentage of coverage may help to ensure sufficient and effective transfer of heat to/from the face and may in particular enable heat transfer with a greater portion of the human face. It may also result in more comfort for the person to whose face the heat transfer unit is applied (herein also referred to as “the wearer”) since any pressure exerted by the heat transfer unit to the face is spread over a greater area.

In one embodiment, the heat transfer unit is sufficiently flexible such that, when applied to a human face, it can be in direct contact with at least 65%, 70%, 75%, 80%, 85% or 90% of the surface of the human face. The percentage of the surface of the face which is in direct contact with the heat transfer unit is expected to be slightly smaller than the percentage of the surface of the face which is covered by the heat transfer unit. This is due to the fact that small pockets of air may remain between the face and the heat transfer unit, for example at the connection locations, where the heat transfer unit may not be in direct contact with the face, given that the first and second flexible material layers effectively meet at the connection locations and “bulge out” between the connection locations. Further, (small) creases may develop where the heat transfer unit is bent so as to adapt to the shape of the face.

In one embodiment, the heat transfer unit has one or more cutouts for one or more of: one or both eyes, a nose and a mouth. In other words, the heat transfer unit doesn't cover these regions. This may enable a wearer to see, breathe, speak, eat and/or drink while wearing the heat transfer unit.

In one embodiment, the heat transfer unit comprises an input port and an output port for connection to a heating or cooling device to allow the heat transfer fluid to flow between the heating or cooling device and the heat transfer unit. The input and output ports can be permanently connected to conduits such as hoses or similar for connection to a heating or cooling device. Alternatively, the input and output ports are suitable for temporary connection to such a conduit. Suitable input/output ports are known in the art and will therefore not be described in detail.

In one embodiment, the fluid chamber has a capacity of less than 300 ml, in particular less than 250 ml or less than 200 ml or less than 180 ml or less than 160 ml or less than 150 ml, in particular wherein the fluid chamber has a capacity of about 140 ml.

The inventor has found that this volume is sufficient to ensure good contact between the heat transfer unit and an average human face. A relatively small volume of the fluid chamber may mean that the capacity of any heating or cooling device to be used with the heat transfer unit can also be reduced accordingly and/or that replenishing of heat transfer fluid during operation becomes unnecessary.

In one embodiment, when the heat transfer unit is used in conjunction with a heat transfer fluid having a temperature of 15° C. in order to cool an average adult face, a surface temperature of the face reaches 22° C. or lower within 15 minutes of operation of the heat transfer unit. This assumes that the heat transfer unit is used in an environment having normal room temperature and humidity.

In one embodiment, the heat transfer unit comprises compartmentalization lines, in particular welding lines or fusion lines, for compartmentalizing the fluid chamber, in particular in order to guide the heat transfer fluid along a desired path through the fluid chamber.

At (or along) the compartmentalization lines, the first and second layers of material are connected to one another, for example using the same or a similar technique as the technique used for connecting the first and second material layers at the connection locations. In particular, the joining of the first and second material layers at the connection locations, along the periphery of the heat transfer unit and along the compartmentalization lines may be performed simultaneously, in particular using the same processing apparatus.

Whilst it is preferred that, at the compartmentalization lines, the first and second layers of material are connected in a fluid tight manner, it would also be acceptable for there not to be a 100% fluid tight seal along the compartmentalization lines. This would simply mean that (a relatively small proportion of) the heat transfer fluid might “take a shortcut” instead of following the desired path. However, the fluid flow across any compartmentalization lines should be significantly smaller than the fluid flow along the desired path, for example no more than 20%, 10% or 5% of the total fluid flow.

In one embodiment, the desired path has several curves or turns, in particular wherein the desired path is a labyrinthine path.

It is envisaged that the width of each individual path section is significantly smaller than the width of the entire heat transfer unit. This may help to ensure that the heat transfer fluid flows along substantially the entire (internal) surface of the transfer unit. By way of contrast, if each individual path section had a relatively large width, the likelihood of the fluid flow not reaching certain areas would increase, for example near the periphery of such a wide path section.

In one embodiment, the heat transfer unit comprises one or more additional layers, in particular an (additional) inner layer and/or an (additional) outer layer, in particular wherein the inner layer is arranged to face towards the human face and/or the outer layer is arranged to face away from the human face, in particular wherein the inner layer and/or the outer layer comprises a fabric layer.

Providing an additional inner layer may make the heat transfer unit more comfortable to wear, in particular if the first and second material layers are made from a plastics material.

An additional outer layer, again for example made from fabric, may help to ensure that one portion of the heat transfer unit can (temporarily) be fastened to another portion, in particular if a fastener such as a hook and loop type fastener is used. To this end, one portion of the heat transfer unit may be provided with the “hook” element of a hook and loop type fastener, and another portion of the heat transfer unit may be provided with the “loop” element of a hook and loop type fastener. Alternatively, the loop element may be omitted if the hook element can (temporarily) attach directly onto the outer layer (such as a fabric layer).

In one embodiment, the heat transfer unit is arranged to be transferred from a substantially flat condition to a bent or folded condition when it is applied to a human face. This enables the heat transfer unit to be manufactured as a flat item, which facilitates manufacture and storage.

For the purpose of the present specification, the expression “folding” (of the heat transfer unit) is not to be understood in the sense of folding a thin piece of paper, which would result in a very sharp edge. Instead, the expression “folding” is to be understood in the sense of bending the heat transfer unit to result in radii of curvature significantly larger than in the case of folding a thin sheet of paper.

In one embodiment, the heat transfer unit comprises a central portion and a plurality of lobes connected to the central portion. The central portion may, for example, occupy an area corresponding to the eye, nose and mouth regions and extending a few centimeters outwardly therefrom (but preferably with cutouts for the eyes, nose and/or mouth, as described above) and may in particular correspond to an area where the face of an average adult human is relatively flat, while the lobes may occupy an area corresponding to the regions extending radially outwardly from the central portion. Providing the heat transfer unit with lobes in the periphery of the heat transfer unit may facilitate bending of the heat transfer unit in places corresponding to facial regions that are less flat, in particular at the forehead and chin regions. Lobes may also be provided laterally, in the cheek regions, where there is a transition from the predominantly forward-facing part of the human face to the predominantly sideways-facing parts of the human face. Alternatively, no such lateral lobes (e.g. in the cheek regions) are provided. In this case, the central portion extends over the entire width of the heat transfer unit, and lobes extend therefrom in an upward direction towards the forehead region and/or in a downward direction towards the chin region.

The shape of the lobes can perhaps be thought of as being somewhat similar to the shape of a petal of a flower such as some types of Ranunculus (also referred to as Buttercup), for example Ranunculus arvensis or Ranunculus repens. In particular, considering each lobe in isolation, only a relatively small portion of its circumference may be connected to the central portion of the heat transfer unit, for example 10% to 40% of the circumference of each lobe. The remainder of the circumference could be “free”, i.e. not (permanently) connected to the central portion or other lobes, so that each lobe can be bent along that part of the circumference of the lobe that is connected (or constitutes the connection) to the central portion whilst such bending substantially does not affect the remainder of the heat transfer unit.

The lobes are not only connected to the central portion but are also in fluid communication therewith, such that the heat transfer fluid can flow not only through the central portion but also through the lobes. However, the flow of heat transfer fluid does not necessarily need to occur over the entire length of the interface between the central portion and a lobe. Instead, barriers or compartmentalization lines may be present at the interface between the central portion and a lobe.

Alternatively, one or more lobes may be connected to the central portion without there also being fluid communication with the central portion.

In one embodiment, neighboring ones of the lobes define a gap therebetween, in particular wherein the gap is arranged to be reduced or substantially closed when the heat transfer unit is bent or folded and applied to a human face. The gap between two lobes may, for example, taper such that the gap is wider at greater distances from the center of the heat transfer unit than at closer distances. When the heat transfer unit is applied to a human face, the size of the gap may reduce, or the gap may close entirely, depending on the size/shape of the head. It is also possible that two lobes would overlap when the heat transfer unit is applied to a human face.

In the present specification, an expression such as “the cheek region of the heat transfer unit” or simply “the cheek region” may be used for the sake of simplicity and is intended to be understood to refer to that region of the heat transfer unit that is intended to cover the cheek region of a human face (of average size and shape). The same applies, mutatis mutandis, to similar expressions referring to portions of the human face (ear region, chin region, forehead region etc.).

In one embodiment, the heat transfer unit further comprises at least one fastening element for temporarily fixing a first one of the lobes to a second one of the lobes or to the central portion. The at least one fastening element may help to keep the heat transfer unit as a whole in contact with the human face. For example, the at least one fastening element may be arranged to extend around the back of the head from a lobe on one side of the heat transfer unit to a lobe on the other side of the heat transfer unit.

In addition, or as an alternative, the at least one fastening element may be arranged to (temporarily) connect one part of the central portion to another part of the central portion, again extending around the (back of the) head.

In addition, or as an alternative, the at least one fastening element may help to keep a particular portion of the heat transfer unit in contact with the human face, in particular (also) in regions where the average human face is curved. To this end, the at least one fastening element may extend from one lobe to (or towards) another lobe (e.g. a neighboring lobe) or to the central portion without extending all the way around the (back of the) head.

In one embodiment, the at least one fastening element is at least partly elastic, in particular wherein the at least one fastening element comprises an elastic portion and a substantially non-elastic portion.

An at least partly elastic fastening element may keep the heat transfer unit more securely in contact with the human face. Alternatively, the at least one fastening element may be substantially non-elastic. Either way, the at least one fastening element may be constructed such that its length can be adjusted and may, for this purpose, comprise a buckle or the like.

In one embodiment, the at least one fastening element comprises a hook-and-loop fastener, in particular wherein a hook portion of the hook-and-loop fastener is arranged to (temporarily) attach to a loop portion permanently attached to the remainder of the heat transfer unit, or wherein a hook portion of the hook-and-loop fastener is arranged to (temporarily) attach directly to a surface of the heat transfer unit.

For example, if the at least one fastening element comprises an elastic portion and a substantially non-elastic portion, the hook portion can be (permanently) attached to the substantially non-elastic portion, and the elastic portion may be located between the substantially non-elastic portion and the remainder of the heat transfer unit.

If the heat transfer unit comprises an additional outer layer, such as a fabric layer, the hook portion may (temporarily) attach directly to that fabric layer, in which case no dedicated loop portion may be necessary.

In one embodiment, the heat transfer unit further comprises a third lobe, wherein the third lobe is located generally between the first lobe and the second lobe, wherein the at least one fastening element for fixing the first lobe to the second lobe is arranged to extend over the third lobe, thereby keeping it in contact with the human face. In this case, the at least one fastening element is neither (permanently) attached to the third lobe, nor is it intended to (temporarily) attach to the third lobe. This may simplify the manufacture of the heat transfer unit when compared with a heat transfer unit that also has a fastening element that is either permanently attached to the third lobe or is intended to attach (temporarily) to the third lobe. Further, it may facilitate applying the heat transfer unit to a human face. Nevertheless, since the at least one fastening element is arranged to extend over the third lobe, the at least one fastening element can also help to keep the third lobe in contact with the face.

In one embodiment, the heat transfer unit has a fastening element in each of the forehead and chin regions, as well as one or two fastening elements to extend around the back of the head (or neck), e.g. one extending from one ear region to the other ear region and one extending from one (lower) cheek region to the other (lower) cheek region.

Fastening elements other than hook and loop fasteners may be used, for example fastening elements that have an adhesive portion for (temporarily) adhering to another surface.

Several sizes of heat transfer units may be provided in order to ensure that a heat transfer unit is available for a relatively large range of face sizes/shapes, in particular not only for adults but also (young) children or even babies. However, the inventor envisages that a single model of heat transfer unit will be suitable for the faces of at least 99% of all adults.

According to a second aspect, the present invention provides a face cuff assembly comprising: a heat transfer unit according to the first aspect or any embodiments or variants thereof; and at least one fastening unit, constructed as one or more devices separate from the heat transfer unit, for fastening the heat transfer unit to a human face.

The at least one fastening unit may be constructed in a substantially similar way as the at least one fastening element described above, except that the fastening unit is provided as a separate item from the heat transfer unit, i.e. is not permanently attached to the heat transfer unit.

In one embodiment, the at least one fastening unit is adjustable, in particular at least partly elastic, and/or
wherein the at least one fastening unit is arranged to extend around the human head and the heat transfer unit.

For example, the at least one fastening unit may comprise a strap of adjustable length, e.g. a strap with a buckle for length adjustment, or may be a strap comprising elastic material. The at least one fastening unit may comprise at least one elastic portion and at least one (substantially) non-elastic portion, or may be formed entirely from elastic material. The at least one fastening unit may extend all the way around the human head (i.e. may extend over a portion of the heat transfer unit and around the back of the head and thus form a loop) or may primarily extend around the back of the head and be arranged to be attached temporarily to the heat transfer unit in at least two places, e.g. in the cheek or ear regions of the heat transfer unit (without extending over the heat transfer unit from one side to the other).

According to a third aspect, the present invention provides a heat transfer apparatus comprising: a heat transfer unit according to the first aspect or any embodiments or variants thereof or a face cuff assembly according to the second aspect or any embodiments or variants thereof;
a heating or cooling device for heating or cooling a heat transfer fluid; and
at least one conduit, in particular two conduits, for connecting the heat transfer unit to the heating or cooling device to allow the heat transfer fluid to flow between the heating or cooling device and the heat transfer unit.

The heating or cooling device would typically be significantly larger than the heat transfer unit and may, for example, be a floor-standing device or a device placed on a stand or table, for example.

According to a fourth aspect, the present invention provides a method of heating or cooling a human face, wherein the method comprises:
providing a heat transfer unit according to the first aspect or any embodiments or variants thereof or a face cuff assembly according to the second aspect or any embodiments or variants thereof or a heat transfer apparatus according to the third aspect or any embodiments or variants thereof;
applying the heat transfer unit to a human face; and
causing a heat transfer fluid to flow into the heat transfer unit, to flow through the fluid chamber and to flow out of the heat transfer unit.

The above steps can be carried out in the stated order or a different order. For example, it is possible to start the fluid flow (i.e. causing the heat transfer fluid to flow into the heat transfer unit, through the fluid chamber and out of the heat transfer unit) before applying the heat transfer unit to the face.

The method of heating or cooling a human face can either be used in the context of a therapeutic treatment, or alternatively for non-therapeutic purposes.

Further advantages, features and applications of the present invention are described in the following detailed description and the appended figures, wherein:

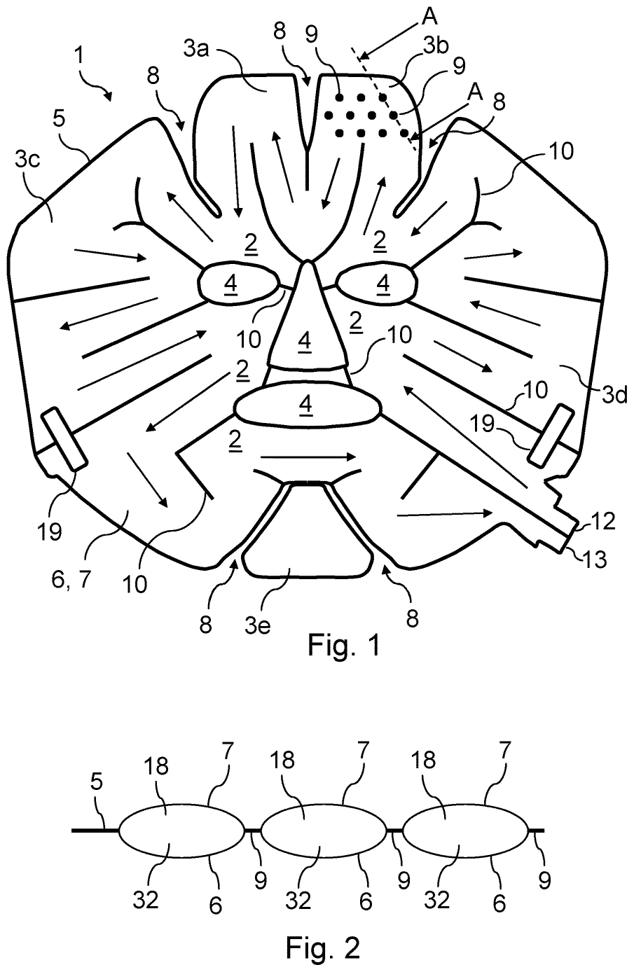

FIG. 1 schematically shows a front view of a heat transfer unit in an unfolded condition in accordance with an embodiment of the present invention.

FIG. 2 schematically shows an enlarged cross-sectional view of a portion of the heat transfer unit of FIG. 1 along line A-A.

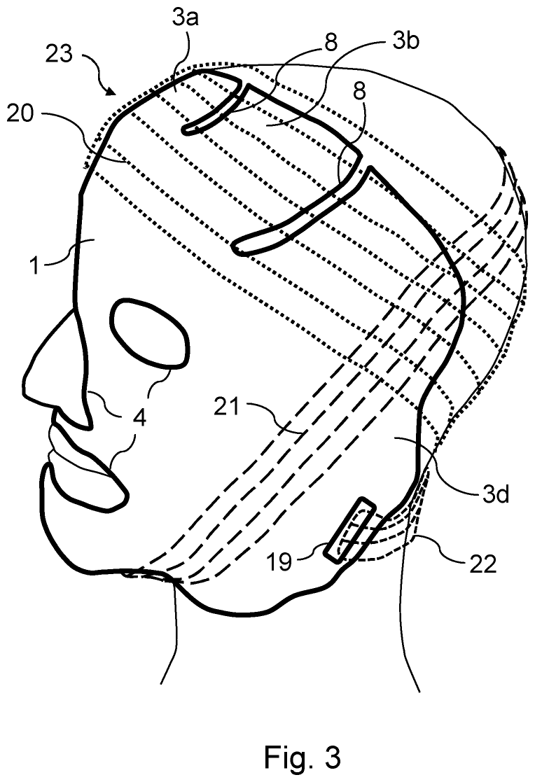

FIG. 3 schematically shows a perspective view of a face cuff assembly applied to a human face/head, in accordance with an embodiment of the present invention.

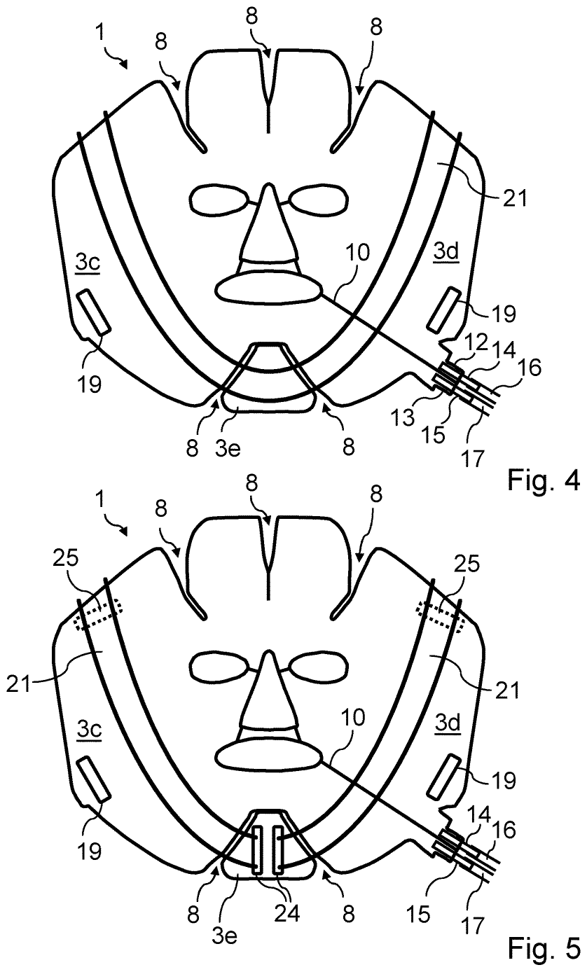

FIG. 4 schematically shows a front view of a heat transfer unit in an unfolded condition in accordance with an embodiment of the present invention.

FIG. 5 schematically shows a front view of a heat transfer unit in an unfolded condition in accordance with an embodiment of the present invention.

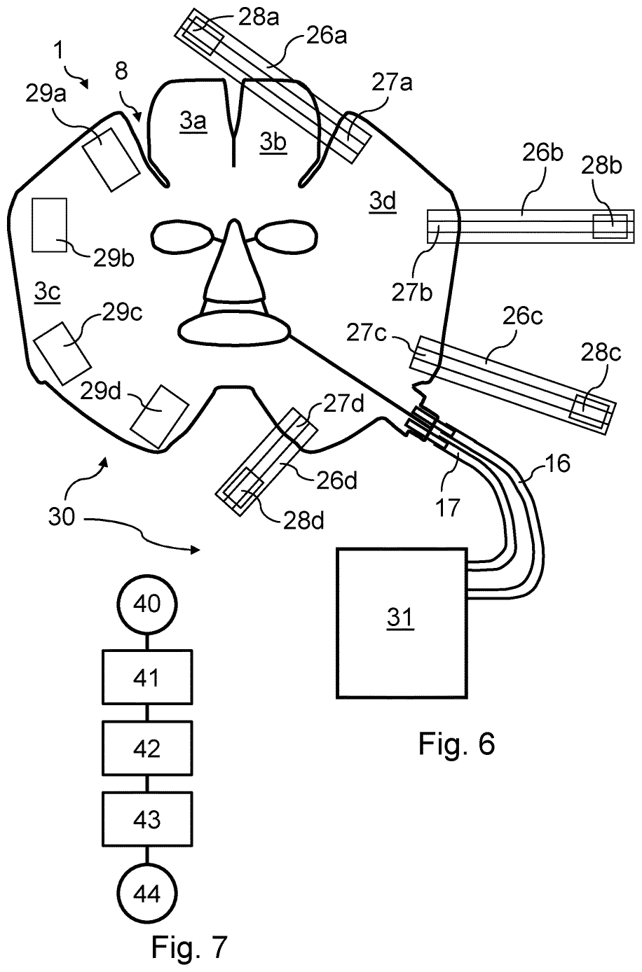

FIG. 6 schematically shows a heat transfer apparatus in accordance with an embodiment of the present invention.

FIG. 7 shows a flowchart illustrating a method in accordance with an embodiment of the present invention.

In the figures, identical reference signs are used for the same or similar elements of the embodiments of the invention described herein. To the extent that an element is shown in more than one figure, this element and its function will not necessarily be explained repeatedly.

FIG. 1 schematically shows a front view of a heat transfer unit 1 (for application to a human face) in an unfolded, substantially flat condition in accordance with an embodiment of the present invention. The heat transfer unit 1 comprises first and second material layers 6, 7, one on top of the other. In use, the first material layer 6 may, for example, face towards the human face and will therefore also be referred to as an inner layer 6. The second material layer 7 may correspondingly face away from the human face and will therefore also be referred to as an outer layer 7. Both material layers 6, 7 are made from a flexible, substantially fluid tight material, for example LDPE or polyurethane. Since FIG. 1 is a front view, the first and second material layers 6, 7 are not shown separately in FIG. 1. The first and second material layers 6, 7 are shown separately in FIG. 2, which schematically shows an enlarged cross-sectional view of a portion of the heat transfer unit 1, along line A-A indicated in FIG. 1. Reference should be made to both figures.

The first and second material layers 6, 7 are connected to one another at their periphery 5, in various connection locations 9 and along compartmentalization lines 10, each of which will be described below. In order to connect the first and second material layers 6, 7 in these locations, the material layers 6, 7 can for example be fused or welded together or adhered to one another using an adhesive. In one example, the two material layers 6, 7 are fused together through the application of pressure and radiation (such as heat), whereby the connections along the periphery 5, at the connection locations 9 and along the compartmentalization lines 10 can be made simultaneously using the same processing apparatus. In locations other than the periphery 5, the connection locations 9 and the compartmentalization lines 10, the first and second material layers 6, 7 remain separate and simply lie one on top of the other, forming passageways 18 therebetween, to enable a heat transfer fluid to flow therethrough. The passageways 18 can have different sizes, in particular depending on the distance between the connection locations 9, the fused periphery 5 and the compartmentalization lines 10, or substantially the same size, as shown in FIG. 2. All passageways together effectively form the fluid chamber 32 bounded by the first and second material layers 6, 7. Whilst the cross-sectional view of FIG. 2 shows the passageways 18 as individual elements, it will be apparent from FIG. 1 that the passageways are (all) connected, given the matrix-like configuration of the individual connection locations 9 in this regard it is noted that FIG. 1 shows a matrix of connection locations only in a small area of heat transfer unit 1, as will be described in more detail below.

It is to be appreciated that the periphery 5 shown in FIG. 1 is not drawn to scale. Whilst FIG. 1 shows periphery 5 as a relatively thin line, the inventor envisages the periphery 5 to be fused over a width of a few millimeters, to ensure a sufficiently secure seal. In order to form a seal at the periphery, the first and second material layers 6, 7 can be placed one on top of the other and then connected as described above, whereby the first and second material layers 6, 7 may either have the size and shape as indicated by the outer edge of periphery 5, or may be larger, in which case the first and second material layers 6, 7 can then be cut to size to result in heat transfer unit 1 having an outline as indicated by periphery 5.

The fused periphery 5 is interrupted in one location—in FIG. 1 this is shown at a position corresponding to 4 o'clock (12 o'clock being towards the top of the figure). This interruption serves as input and output ports 12, 13. Conduits (not shown in FIG. 1, but described below) can be connected to the input port 12 and the output port 13 so that heat transfer fluid can be supplied into, and removed from, the heat transfer unit 1. Several arrows generally indicate the direction of flow of the heat transfer fluid (or a desired fluid path) through the fluid chamber 32 of the heat transfer unit 1.

The fluid path has several turns or curves, resulting in a serpentine pattern or labyrinthine path. The fluid path is primarily defined by the inner edge of the periphery 5 and a number of compartmentalization lines 10. Some of the compartmentalization lines 10 are joined onto the periphery 5 of the heat transfer unit 1, whilst some others—located more towards the middle of FIG. 1—are not. Some of the compartmentalization lines 10 are branched.

The compartmentalization lines 10 help to ensure that the heat transfer fluid flows across substantially the entire surface of the heat transfer unit 1. The width of each section of the fluid path formed by the compartmentalization lines 10 and the periphery 5 is significantly smaller than the overall width of the heat transfer unit 1.

Whilst the input and output ports 12, 13 are shown in a particular location of the periphery 5, the input and output ports 12, 13 could also be provided at a different location, either at a different location along the periphery 5 or even within (only) one of the first and second material layers 6, 7 (i.e. not integrated into the periphery 5). Further, the arrangement of the compartmentalization lines 10 as shown in FIG. 1 is only one example. A different arrangement of compartmentalization lines 10, perhaps with more, or fewer compartmentalization lines 10, is also conceivable. Also, the direction of flow of the heat transfer fluid along the fluid path could be reversed by using port 13 as the input port and using port 12 as the output port.

As indicated in FIG. 1, the heat transfer unit 1 generally has a central portion 2, as well as several lobes 3a-3e extending therefrom, generally in a radially outward direction. The central portion 2 is indicated in FIG. 1 in several exemplary locations using reference sign 2. Four cutouts 4 are provided within the central portion 2, respectively for the two eyes, nose and mouth of a wearer of the heat transfer unit 1, i.e. the eyes, nose and mouth are not covered by the heat transfer unit 1 when the heat transfer unit 1 is applied to a wearer's face. As an alternative, one or more of the cutouts could be omitted, for example in the eye and mouth regions, so that only a cutout 4 for the nose remains. The compartmentalization lines 10 would then need to be rearranged accordingly, to ensure that the heat transfer fluid flows along a desired path. The first and second material layers 6, 7 are connected to one another along the edge of the cutouts 4 to ensure that the fluid chamber 32 is sealed in those regions as well.

In the example shown in FIG. 1, some compartmentalization lines 10 extend from the eye and mouth regions 4 radially outwardly. Further, relatively short compartmentalization lines 10 are provided between the nose region 4 and each eye region 4, as well as between the nose and mouth regions 4 (below the left-hand and right-hand edges of the nose region 4). The first and second material layers 6, 7 can be connected (e.g. fused together) over the entire area between the nose and mouth regions 4 (the area including the philtrum).

The heat transfer unit 1 shown in FIG. 1 has two further cutouts or openings 19 for securing the heat transfer unit 1 to a human face. This will be described in more detail below.

In the embodiment shown in FIG. 1, five lobes are provided: two lobes 3a, 3b, respectively for the left and right half of the forehead region, two lobes 3c, 3d, respectively for the left and right cheek and ear regions, as well as a lobe 3e for the chin region. The two lobes 3c, 3d for the cheek and ear regions are significantly larger than the other three lobes 3a, 3b, 3e.

A gap 8 is formed between each pair of neighboring lobes. At least the three gaps in the forehead region are wider at positions that are radially more outward than at positions that are radially more inward. When the heat transfer unit 1 is applied to a human face, the lobes would be bent into contact with the human face, i.e. the two forehead lobes 3a, 3b would be bent to follow the shape of the forehead, the cheek/ear lobes 3c, 3d would be bent around the sides of the face and the chin lobe would be bent to follow the shape of the chin, whereby chin lobe 3e may overlap the bottom ends of lateral lobes 3c, 3d. As the lobes are bent, the gaps 8 become narrower than when the heat transfer unit 1 is in the flat condition shown in FIG. 1. Depending on the size and shape of the face to which the heat transfer unit 1 is applied, the gaps 8 may close entirely, or neighboring lobes may even overlap.

Whilst several definitions are possible as to where the central portion 2 stops and where lobes 3a-3e start, the central portion 2 can generally be regarded as the area in which the cutouts 4 are located, as well as an area surrounding the cutouts 4 and extending a few centimeters (e.g. about 2 to 3 cm) outwardly. The central portion 2 can, for example, be regarded as an approximately circular or oval region that extends up to, but does not include, the gaps 8, i.e. approximately corresponding to the forward-facing region of a human face.

The inventor has found that satisfactory or good results can be achieved with a heat transfer unit 1 generally shaped as shown in FIG. 1. However, in other embodiments, the size and shape of the lobes 3a-3e and of the gaps 8 may be different from those shown in FIG. 1. Further, other embodiments are possible with more than five lobes (e.g. where the lobes 3c and 3d are each subdivided into two smaller lobes), or with fewer than five lobes (e.g. where lobes 3a and 3b are combined into a single forehead lobe).

In the example shown in FIG. 1, the lobes 3a-3d are not only joined onto the central portion 2 but are also in fluid communication therewith, so that the heat transfer fluid can flow not only through the central portion 2 but also through the lobes 3a-3d. The chin lobe 3e is however not in fluid connection with the central portion 2—the inventor has found that such a fluid connection between the central portion 2 and the chin lobe 3e is in many cases not necessary since, when the cheek lobes 3c, 3d are bent rearwards into contact with the lateral portions of the human face, the lower parts of cheek lobes 3c, 3d tend to meet under the chin so that heat transfer between the heat transfer unit 1 and the lower chin region is achieved by the cheek lobes 3c, 3d. Chin lobe 3e would then normally simply rest on the lower parts of cheek lobes 3c, 3d. Of course, it would also be possible to provide a fluid connection between the central portion 2 and the chin lobe 3e.

The central portion 2 and the lobes 3a-3e are provided with connection locations 9, where first and second material layers 6, 7 are connected to one another. In FIG. 1, connection locations are indicated only in a small portion of the heat transfer unit 1 so as not to obscure other details. However, a corresponding pattern of connection locations 9 can be present throughout the heat transfer unit 1. In the example shown in FIG. 1, three nearest neighbors of connection locations 9 form an equilateral triangle. In other variants, other regular patterns are possible, including a pattern where four nearest neighbors of connection locations 9 form a square. In yet other variants, the pattern may be an irregular/random pattern. Further, different regions of the heat transfer unit 1 may have different patterns of connection locations 9.

FIG. 3 schematically shows a perspective view of a face cuff assembly 23 applied to a human face/head, in accordance with an embodiment of the present invention.

In the example shown, the face cuff assembly 23 comprises a heat transfer unit, such as the heat transfer unit 1 described above (or any of its variants) and one or more fastening units 20, 21, 22, such as bandages or straps 20, 21, 22 for securing the heat transfer unit 1 to the human face/head. In FIG. 3, the outline of the human face/head is drawn in a thin continuous line, the outline of the heat transfer unit 1 is drawn in a thick continuous line, a first bandage 20 is indicated by dotted lines, a second bandage 21 is indicated by dashed lines with long dashes, and a strap 22 is indicated by dashed lines with short dashes.

For the sake of clarity, much of the detail of the heat transfer unit 1 shown in FIG. 1 has been omitted from FIG. 3, such as the connection locations 9, the compartmentalization lines 10 and the input and output ports 12, 13.

The heat transfer unit 1 is applied to the face such that the eyes, nose and mouth of the face are approximately aligned with the cutouts 4. The lobes 3a-3e are bent backwards so as to approximately follow the shape of the head. This causes the width of the gaps 8 to narrow or substantially close.

The bandages and straps 20, 21, 22 may be elastic or adjustable, for example by means of a buckle or similar (not shown), and are indicated by several approximately parallel lines. Bandages 20 and 21 extend over the heat transfer unit 1 in order to keep it in contact with the face. Bandage 20 extends over the forehead region so as to keep lobes 3a, 3b in place, as well as the top portion of lobes 3c and 3d. Bandage 20 also extends around the back of the head. Bandage 21 extends over chin lobe 3e (not shown in FIG. 3) and the lower end of lobes 3c (not shown in FIGS. 3) and 3d. It then extends over the cheek and ear regions and around the back of the head, at a higher position than bandage 20. Strap 22 passes through opening 19 and extends around the back of the neck and passes through a corresponding opening 19 on the other side of the neck.

Bandages 20, 21 are provided as separate items from heat transfer unit 1. Strap 22 can also be provided as a separate item from heat transfer unit 1 and, for example, form a loop with a buckle or a hook and loop fastening arrangement for closing the loop. Alternatively, strap 22 can be (permanently) connected to the heat transfer unit 1. In this case, one end of strap 22 can pass through one opening 19 and be folded back and secured to an intermediate portion of strap 22, for example by sewing. The other (free) end of strap 22 can pass through the other opening 19 on the opposite side of heat transfer unit 1 and temporarily be attached to an intermediate portion of strap 22, for example with a hook and loop fastening arrangement. In this context, it is to be noted that a dedicated loop element can be omitted if the hook element (e.g. permanently attached to the free end of strap 22) can attach directly to an intermediate portion of strap 22. FIG. 4 schematically shows a front view of a heat transfer unit 1 in an unfolded condition in accordance with an embodiment of the present invention. The heat transfer unit 1 shown in FIG. 4 corresponds to the heat transfer unit 1 shown in FIGS. 1 and 3. For the sake of clarity, much of the detail of the heat transfer unit 1 shown in FIG. 1 has again been omitted from FIG. 4, such as the connection locations 9 and most of the compartmentalization lines 10. Instead, FIG. 4 indicates an approximate possible position of bandage 21, i.e. it indicates how bandage 21 might extend over the heat transfer unit 1 when applied to a face. As described above in connection with FIG. 3, it is envisaged that lobes 3c and 3d would be bent into contact with the cheek and ear regions of the face, whereby the lower end of lobes of 3c and 3 d would also come into contact with the chin region, in particular the region under the chin. Lobe 3e would then be bent backwards, overlapping lobes 3c and 3d. When applied to the face, bandage 21 would extend over lobe 3e, then over lobe 3d, then around the back of the head and then over lobe 3c. It should be noted that FIG. 4 shows one example of a bandage 21, in particular as regards its width and position. The position of bandage 21 can be adjusted to result in a secure fit and good contact between heat transfer unit 1 and the face. Further, bandages of different widths can be chosen.

FIG. 4 also shows a portion of an input hose 16, which is connected to input port 12 via an input connector 14. Similarly, FIG. 4 shows a portion of an output hose 17, which is connected to output port 13 via an output connector 15. Suitable hoses and connectors for connection to input and output ports 12, 13 are known to one skilled in the art and will therefore not be described in detail. In any event, other input and output arrangements are possible. Input and output hoses 16, 17 are for connection to a heating or cooling device, as will be described below.

In a variant to the embodiment shown in FIG. 4, the chin lobe 3e can be omitted. When the heat transfer unit 1 is applied to a face, the lateral lobes 3c, 3d would reduce or substantially close the gap 8 in the chin region (or even overlap), and bandage 21 would then pass over the lower end of lobes 3c and 3d to retain these in contact with the chin region, without there being a chin lobe 3e overlapping the lower end of lobes 3c and 3d.

FIG. 5 schematically shows a front view of a heat transfer unit in an unfolded condition in accordance with an embodiment of the present invention, which can be regarded as a variant of the embodiment shown in FIG. 4. Compared with FIG. 4, the bandage 21 shown in FIG. 5 does not simply pass over chin lobe 3e. Instead, lobe 3e is provided with two further openings or cutouts 24, and bandage 21 passes through both openings 24. That is, starting in the left half of the figure, bandage 21 would pass over lateral lobe 3c, then over the left-hand portion of chin lobe 3e, then through the left-hand opening 24, then pass behind the middle portion of lobe 3e, then through the right-hand opening 24, then over the right-hand portion of lobe 3e and then over lateral lobe 3d. Apart from that, bandage 21 can pass around the back of the head in the same way as explained above in connection with FIG. 4.

FIG. 5 indicates a further variant. This variant has two further openings or cutouts 25 (shown in FIG. 5 in dotted lines) near the top end of lateral lobes 3c, 3d. In this variant, bandage 21 would extend over heat transfer unit 1 as described above, except that, near the top end of lateral lobes 3c, 3d, bandage 21 would pass through openings or cutouts 25 and then extend behind the top end of lateral lobes 3c, 3d.

It will be appreciated that the openings 24 and/or 25 may help to better locate bandage 21 in relation to the heat transfer unit 1 and may therefore keep the heat transfer unit 1 more securely in place when applied to a human face.

FIG. 6 schematically shows a heat transfer apparatus 30 in accordance with an embodiment of the present invention. The heat transfer apparatus 30 comprises a heat transfer unit 1, a heating or cooling device 31 for heating or cooling a heat transfer fluid, and connecting hoses 16, 17 between the heating or cooling device 31 and the heat transfer unit 1, in particular an input hose 16 and an output hose 17, respectively for enabling the heat transfer fluid to flow into, and out of, the heat transfer unit 1. The heating or cooling device 31 is not shown to scale-it would typically be significantly bigger than heat transfer unit 1. Similarly, the hoses 16, 17 would typically be significantly longer than the width of the heat transfer unit 1.

The heat transfer unit 1 to be used in conjunction with heating or cooling device 31 and hoses 16, 17 may be as any one described above, or alternatively as the heat transfer unit 1 schematically illustrated in FIG. 6. Instead of being provided with bandages or straps 20, 21, 22 which are either entirely separate items from heat transfer unit 1 and/or which pass through openings/cutouts of heat transfer unit 1, the heat transfer unit 1 of FIG. 6 has several straps 26a-26d, which are substantially permanently attached to heat transfer unit 1, for example sewn onto heat transfer unit 1. In the example shown in FIG. 6, four such straps 26a-26d are provided: strap 26a is attached to the top end of lateral lobe 3d, strap 26b is attached to the ear region of lateral lobe 3d, strap 26c is attached to the lower cheek region of lateral lobe 3d, and strap 26d is attached to the bottom end of lateral lobe 3d, in the chin region. The attached ends 27a-27d of straps 26a-26d, i.e. those ends of straps 26a-26d which are permanently attached to heat transfer unit 1, can, for example, be sewn directly onto the fused periphery 5 of heat transfer unit 1. Another way of fixing straps 26a-26d involves the heat transfer unit 1 having at least one additional layer, for example an outer fabric layer (not shown), onto which straps 26a-26d are sewn.

The free ends 28a-28d of straps 26a-26d are intended for temporary attachment to a portion of heat transfer unit 1, for example where indicated by four rectangles 29a-29d. To this end, a hook and loop fastening arrangement can be employed. For example, the free ends 28a-28d can be provided with a hook element (e.g. sewn onto straps 26a-26d), and a loop element can be provided, approximately where indicated by the four rectangles 29a-29d, in particular substantially permanently attached thereto, for example by sewing loop elements 29a-29d onto an outer fabric layer of the heat transfer unit 1, or attaching loop elements 29a-29d directly onto outer layer 7, for example using an adhesive.

When the heat transfer unit 1 is applied to a face, the lobes 3a-3e are first bent into contact respectively with the forehead, ear, cheek and chin regions. The free ends 28a-28d of straps 26a-26d are then attached to loop elements 29a-29d: Strap 26a is applied over lobes 3a and 3b and partly over the top end of lobe 3c, with hook element 28a attaching to loop element 29a. Strap 26d is applied partly over the bottom end of lobe 3c, with hook element 28d attaching to loop element 29d. Strap 26b is extended around the back of the head of the wearer and partly over the ear region of lobe 3c, with hook element 28b attaching to loop element 29b. In this context it is noted that strap 26b would be significantly longer than what is shown in FIG. 6. In particular, given that strap 26b would need to extend around the back of the head of the wearer, strap 26b would normally be longer than the other straps 26a, 26c, 26d. Strap 26c is extended around the top end of the neck of the wearer and partly over the cheek region of lobe 3c, with hook element 28c attaching to loop element 29c.

It is worth noting that strap 26a can retain forehead lobes 3a and 3b in contact with the forehead without lobes 3a and 3b themselves being provided with a fastener since strap 26a extends over lobes 3a and 3b when strap 26a is attached to the top end of lobe 3c where indicated by reference numeral 29a.

Whilst a particular embodiment has been illustrated in FIG. 6, it should be noted that a number of variations are possible:
1. Instead of straps 26 a-26d being provided with hook elements 28 a-28d and loop elements 29a-29d being provided on lateral lobe 3c, the positions of some or all of the hook and loop elements could be swapped so that hook elements are provided on lateral lobe 3c, with loop elements being provided on straps 26a-26d.
2. Instead of loop elements being provided (either on lateral lobe 3 c or on straps 26 a-26d), any hook element provided could also attach directly to lateral lobe 3c or to the straps 26a-26d, provided they are of a suitable material or covered with a suitable material (e.g. fabric).
3. Instead of the hook and loop fastening arrangement, straps 26a-26d could alternatively be applied temporarily to lateral lobe 3c in some other way, for example using a (temporary) adhesive.
4. Some or all of the straps 26a-26d could be (permanently) attached to lateral lobe 3c, for temporary attachment to lateral lobe 3d.
5. As described in connection with strap 22 (cf. FIG. 3), the straps 26a-26d can be elastic or at least partly elastic. For example, a region at one or both ends of the straps 26a-26d (i.e. where indicated by reference signs 27a-27d and/or 28a-28d) could be substantially inelastic, with the remaining portion of straps 26a-26d being made from an elastic material.
6. Any, some or all of straps 26a-26d may be adjustable in length and for this purpose may be provided with a buckle or similar.

More generally, individual features of the embodiments described can be used in other combinations. For example, a bandage 20 (cf. FIGS. 3 to 5) could be used instead of straps 26a and 26b to keep heat transfer unit 1 in contact with the forehead and ear regions, while straps 26c and 26d (cf. FIG. 6) are used to keep heat transfer unit 1 in contact with the cheek and chin regions.

FIG. 7 shows a flowchart illustrating a method in accordance with an embodiment of the present invention. After the start 40 of the method, a heat transfer unit (such as heat transfer unit 1 described above), a face cuff assembly (such as face cuff assembly 23 described above and comprising a heat transfer unit) or a heat transfer apparatus (such as heat transfer apparatus 30 described above and comprising a heat transfer unit) is provided in step 41. In step 42, the heat transfer unit is applied to a human face in the manner described above. In step 43, a heat transfer fluid is caused to flow into the heat transfer unit, to flow through the fluid chamber and to flow out of the heat transfer unit. Thereafter, the method can end (step 44). The method can be used for therapeutic purposes, or exclusively for non-therapeutic purposes.

While at least one example embodiment of the present invention has been described above, it has to be noted that a great number of variations thereto exist. Furthermore, it is to be appreciated that the described example embodiments only illustrate non-limiting examples of how the present invention can be implemented and that it is not intended to limit the scope, the application or the configuration of the apparatuses and methods described herein. Rather, the preceding description will provide the person skilled in the art with instructions for implementing at least one example embodiment of the invention, whereby it has to be understood that various changes in the functionality and the arrangement of the elements of the example embodiment can be made without deviating from the subject-matter defined by the appended claims and their legal equivalents.

### LIST OF REFERENCE SIGNS

1 heat transfer unit
2 central portion
3a-3e lobes
4 cutouts
5 periphery/fused portion
6 first material layer
7 second material layer
8 gap
9 connection locations, (welding/fusion) dots
10 compartmentalization lines, (welding/fusion) lines
12 input port
13 output port
14 input connector
15 output connector
16 input hose
17 output hose
18 passageways
19 opening/cutout
20-22 bandages/straps
23 face cuff assembly
24, 25 opening/cutout
26a-26d strap
27a-27d attached end of strap
28a-28d free end of strap/hook element
29a-29d loop element
30 heat transfer apparatus
31 heating or cooling device
40-44 method steps

1. A heat transfer unit adapted to be applied to a human face in order to transfer heat to/from the human face,
wherein the heat transfer unit comprises a first flexible material layer and a second flexible material layer defining a fluid chamber therebetween, allowing a heat transfer fluid to flow through the fluid chamber,
wherein the first and second flexible material layers are connected to one another at a plurality of connection locations,
wherein the heat transfer unit is dimensioned such that, when applied to a human face, it can cover at least 70% of the surface of the human face.

2. The heat transfer unit according to claim 1, wherein the heat transfer unit is dimensioned such that, when applied to a human face, it can cover at least 75%, 80%, 85% or 90% of the surface of the human face.

3. The heat transfer unit according to claim 1, wherein the heat transfer unit is sufficiently flexible such that, when applied to a human face, it can be in direct contact with at least 65%, 70%, 75%, 80%, 85% or 90% of the surface of the human face.

4. The heat transfer unit according to claim 1, wherein the heat transfer unit has one or more cutouts for one or more of:
one or both eyes
a nose
a mouth.

5. The heat transfer unit according to claim 1, wherein the heat transfer unit comprises an input port and an output port for connection to a heating or cooling device to allow the heat transfer fluid to flow between the heating or cooling device and the heat transfer unit.

6. The heat transfer unit according to claim 1, wherein the heat transfer unit comprises compartmentalization lines, in particular welding lines or fusion lines, for compartmentalizing the fluid chamber, in particular in order to guide the heat transfer fluid along a desired path through the fluid chamber.

7. The heat transfer unit according to claim 6, wherein the desired path has several curves or turns, in particular wherein the desired path is a labyrinthine path.

8. The heat transfer unit according to claim 1, wherein the heat transfer unit is arranged to be transferred from a substantially flat condition to a bent or folded condition when it is applied to a human face.

9. The heat transfer unit according to claim 1, wherein the heat transfer unit comprises a central portion and a plurality of lobes connected to the central portion.

10. The heat transfer unit according to claim 9, wherein neighboring ones of the lobes define a gap therebetween, in particular wherein the gap is arranged to be reduced or substantially closed when the heat transfer unit is bent or folded and applied to a human face.

11. The heat transfer unit according to claim 9, further comprising at least one fastening element for temporarily fixing a first one of the lobes to a second one of the lobes or to the central portion.

12. The heat transfer unit according to claim 11, wherein the at least one fastening element is at least partly elastic, in particular wherein the at least one fastening element comprises an elastic portion and a substantially non-elastic portion.

13. The heat transfer unit according to claim 11, wherein the at least one fastening element comprises a hook-and-loop fastener, in particular wherein a hook portion of the hook-and-loop fastener is arranged to attach to a loop portion permanently attached to the remainder of the heat transfer unit, or wherein a hook portion of the hook-and-loop fastener is arranged to attach directly to a surface of the heat transfer unit.

14. The heat transfer unit according to claim 11, wherein the heat transfer unit further comprises a third lobe, wherein the third lobe is located generally between the first lobe and the second lobe, wherein the at least one fastening element for fixing the first lobe to the second lobe is arranged to extend over the third lobe, thereby keeping it in contact with the human face.

15. A face cuff assembly comprising:
a heat transfer unit according to claim 1; and
at least one fastening unit, constructed as one or more devices separate from the heat transfer unit, for fastening the heat transfer unit to a human face.

16. The face cuff assembly according to claim 15, wherein the at least one fastening unit is adjustable, in particular at least partly elastic, and/or
wherein the at least one fastening unit is arranged to extend around the human head and the heat transfer unit.

17. A heat transfer apparatus comprising:
a heat transfer unit according to claim 1 or a face cuff assembly according to claim 16;
a heating or cooling device for heating or cooling a heat transfer fluid; and
at least one conduit, in particular two conduits, for connecting the heat transfer unit to the heating or cooling device to allow the heat transfer fluid to flow between the heating or cooling device and the heat transfer unit.

18. A method of heating or cooling a human face, wherein the method comprises:
providing a heat transfer unit according to claim 1 or a face cuff assembly according to claim 15 or a heat transfer apparatus according to claim 17; applying the heat transfer unit to a human face; and
causing a heat transfer fluid to flow into the heat transfer unit, to flow through the fluid chamber and to flow out of the heat transfer unit.
